(19) United States
(12) Patent Application Publication
(10) Pub. No.: US 2016/0310623 A1
(43) Pub. Date: Oct. 27, 2016

(54) Transparent Melt

(71) Applicant: Belle-Aire Fragrances, Inc., Mundelein, IL (US)

(72) Inventors: Christopher Kelly, Chicago, IL (US); Richard L. Johnson, Racine, WI (US)

(21) Appl. No.: 15/071,868

(22) Filed: Mar. 16, 2016

Related U.S. Application Data
(60) Provisional application No. 62/150,396, filed on Apr. 21, 2015.

(51) Int. Cl. A61L 9/012; A61L 9/02

(52) U.S. Cl. CPC A61L 9/012; A61L 9/02

(57) ABSTRACT

A transparent melt is described, comprising a combination comprising at least one polyamide polymer, at least one carrier solvent, and a polarity-adjusted additive. The additive can be at least one of a fragrance, a mal-odor counteractant, a therapeutic agent, an insect repellant, a pesticide, and mixtures thereof for volatizing into the air.

### FIELD OF THE INVENTION

This invention is directed to a transparent melt for use in a warming device. The melt can contain an additive such as a fragrance, a mal-odor counteractant, a therapeutic agent, an insect repellant, a pesticide, an air freshener, or mixtures thereof, for volatizing into the air.

### BACKGROUND OF THE INVENTION

Candles made of conventional waxes are well known. A common type of candle that sees widespread use consists of a wick in a block of petroleum wax. The wax provides the fuel for the burning candle. Additives such as a fragrance, a therapeutic agent, a pesticide, or an air freshener can he embedded in the wax.

These candles have limited aesthetics and are opaque in nature. More recently, transparent candies have evolved. For example, U.S. Pat. No, 6,111,055 to Berger et al. described the use of candles derived from ester-terminated polyamide [“ETPA”] gels. U.S. Pat. No. 5,879,694 describes candles comprising a hydrocarbon oil, a wick, and one or more triblock, radial block or multiblock copolymers of a thermoplastic rubber. These candles can have an additive such as a fragrance, a therapeutic agent, a pesticide, or an air freshener embedded in the fuel material.

All candles, however, suffer from several shortcomings in today's environmentally challenged world. First, additive delivery is critically important to candle success. Candles are limited to less than 12 percent fragrance, however, because of stability and safety concerns and are very inefficient in delivering scent because a high percentage of the fragrance is consumed in the candle flame. Additionally, candles produce soot which is not beneficial to the breathable environment. Moreover, candle flames are seen as fire safety hazards by consumers.

One alternative to candles is the use of wax melts, sometimes referred to as wax tarts. An additive such as a fragrance is mixed with a wax base, usually a paraffin or vegetable wax. The wax melt is placed in a warming device that heats the material above its melt point, thereby releasing the additive. Usually, the heat source is electric, although use of a flame is possible as well. A. diagram of a prior-art candle warmer appliance 20 is shown in FIGS. 1 and 2. A wax melt 22 is placed in a container 24 of appliance 20, as shown in FIG. 1. A heat source 26 warms container 24, melting wax melt 22, as shown in FIG, 2. In this example, heat source 26 is a heat element connected by power cord 28 to a standard household electrical outlet. (Alternatively, a battery could be used.) Once wax melt 22 has liquefied, it releases to atmosphere any additive in wax melt 22. If the additive is, for example, a fragrance, the fragrance is released to atmosphere and provides a pleasing scent to nearby persons.

The use of wax melts as a form of additive delivery still suffers from limiting factors, such as a waxy opaque appearance, a petroleum smell, and a fragrance limit of about 15 percent, Use of fragrance at high levels can cause stability issues such as syneresis of the material. Syneresis is the spontaneous separation of a liquid from a melt due to factors such as aging, temperature fluctuation, and contraction of the material. There is a marketing advantage to having low or zero syneresis, as a melt that has separated will present an unattractive appearance to consumers. Furthermore, the messy removal of spent wax from the warmer appliance and the non-reusability of the spent wax are unattractive features to consumers,

Additionally, wax melts are opaque. Consumers tend to prefer transparent candle fuels so there is a marketing advantage to having a transparent material. There is also a preference from consumers for translucent materials in this area, materials which are transparent but having a color indicative of the additive. For example, consumers tend to associate red with spice, green with mint, and yellow or orange with fruit, and will prefer candle fuels having a fragrance associated with the color. Additionally, consumers associate colors with holidays, and will prefer candle fuels tinted red for Christmas, green for St. Patrick's Day, orange for Halloween, etc. Moreover, consumers associate colors with functional additives, and will prefer candle fuels tinted green, for example, if there is a pesticide additive such as a mosquito repellant. Accordingly, there is a marketing advantage to having a candle fuel that is transparent but tinted with a color.

Additionally, the suspension of inert materials such as glitter into a transparent melt can produce a further decorative effect that provides a marketing advantage.

The present invention ameliorates the problems of the prior art and provides a product tailored to consumer preferences.

### SUMMARY OF THE INVENTION

The preferred embodiment of the method of the present invention is a transparent melt comprising at least one polyamide resin, at least one carrier solvent designed to adjust the polarity of the transparent melt, and a polarity-adjusted additive. Another embodiment comprises a combination of a warming device and a transparent melt of the preferred embodiment. The additive comprises one or more of a fragrance, a mal-odor counteractant, a therapeutic agent, an insect repellant, and a pesticide or mixtures thereof for volatizing into the air.

### BRIEF DESCRIPTION OF THE DRAWINGS

The organization and Manner of the structure and operation of the invention, together with further objects and advantages thereof, may best be understood by reference to the following description, taken in connection with the accompanying non-scale drawings, wherein like reference numerals identify like elements in which:

FIG. 1 is a warming device as known in the prior art, with an unmelted wax melt. placed in the container.

FIG. 2 is the warming device of FIG. 1, with the wax melt in liquid state.

### DETAILED DESCRIPTION OF THE ILLUSTRATED EMBODIMENTS

While the invention may be susceptible to embodiment in different forms, there is shown in the drawings, and herein will be described in detail, specific embodiments with the understanding that the present disclosure is to be considered an exemplification of the principles of the invention, and is not intended to limit the invention to that as illustrated and described herein.

The preferred embodiment comprises a melt for use in a melt warming appliance such as the one shown in FIG. I, preferably one capable of producing temperatures of 125 F to 200 F, more preferably one capable of producing temperatures of 150 F to 200 F. The melt of the preferred embodiment is thermally reversible and will preferably hold from one to 60 percent additive, more preferably 15 percent to 40 percent additive, even more preferably about 25 percent additive. The preferred embodiment of the present invention will be described with a polarity-adjusted fragrance as an additive. The additive can be another material, however, including but not limited to a malodor counteractant, a therapeutic agent, an insect repellant, a pesticide, or an air freshener, or mixtures thereof, for volatizing into the air. Thus, the preferred embodiment comprises at least one polyimide polymer, at least one carrier solvent, and an additive. In another embodiment, a dye is added for translucence. In yet another embodiment, an inert material such as glitter is added.

It is a goal of the invention to have a material that is transparent or translucent. The inventors have found that the use of a polyamide polymer and at least one carrier solvent with a polarity-adjusted fragrance will produce a material with very low haze. Surprisingly, the manipulation of proportions of carrier solvents contributes to achieving this goal.

The degree of translucence or “haze” of a material is the cloudiness of a transparent material that is caused by scattering of light through the transparent material. Haze is responsible for a cloudy appearance of the material. Fragrances tend to cause a carrier solvent to appear hazy. A hazy material is difficult to market, as consumers tend to prefer transparent materials. The inventors have found that a delicately-balanced mixture of fragrance with non-polar and polar carriers will achieve haze values of five percent or less as measured by ASTM D1001-13 (Standard Test Method for Haze and Luminous Transmittance of Transparent Plastics) when measured on, for example, a Hunter Lab spectrophotometer. This standard test measures the percent of transmitted light that is scattered more than 2.5 degrees from the direction of an incident beam. A delicate balance of carrier solvents, besides producing the desired clarity, also achieves a melt index between 125 F and 200 F, more preferably between 150 F and 200 F, and allows the incorporation of fragrances in the one to 60 percent by weight range.

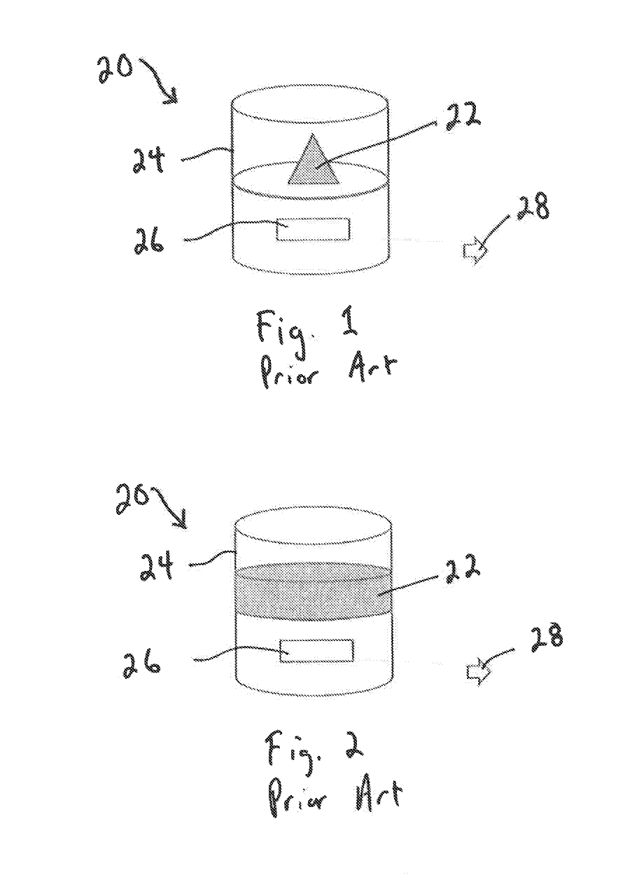

A melt index greater than about 12.5 F allows the product to be used in a home warming device such as the appliance of FIG. 1. Devices on the market today generally warm the candle fuel to 125 F to 200 F. A melt index lower than about 125 F might cause melting at ambient temperatures found in a residential home, patio, or yard during summer months, in direct sunlight, or in a truck during transportation to retail stores. A melt index higher than this range will cause safety issues in a home or office and will cause performance issues.

Recent advances in solubility theory have improved the accuracy of predictions using solubility parameters. A solubility parameter is the sum of all the cohesive forces and the square root of the energy of vaporization. See Henderson, JOURNAL OF THE SOCIETY OF COSMETIC CHEMIST (September/October 1985) at Table I. Applying these parameters, the inventors have found that the most preferred range of solubility are materials having a solubility parameter between that of white mineral oil (7.09) and castor oil (8.9).

Another important factor is syneresis, One surprising result of the present invention is the lack of any significant syneresis even at high fragrance loads of up to 60 percent,

The polyamide of the present invention is preferably an ETPA, a tertiary-terminated polyamide [“ATPA”], a polyalkyleneoxy-terminated polyamide [“PAOPA”], or a polyether polyamine [“PEP”]. The polyamide should have a molecular weight between about 4,700 and 20,000, a color of less than eight on the Gardner Color Scale (PAINT AND COATING TESTING MANUAL: GARDNER-SWARD HANDBOOK (15^{th} ed,)), an acid number of less than 20, as measured by ASTM D664-11a (Standard Test Method for Acid Number of Petroleum Products by Potentiometric Titration), and an amine number of less than three, as measured by ASTM D2074-07(2013) (Standard Test Methods for Total, Primary, Secondary, and Tertiary Amine Values of Fatty Amines by Alternative Indicator Method). The inventors have found that polyamides sold under the brand names CrystaSense LP1, CrystaSense LP2, CrystaSense LP3, CrystaSense MP, CrystaSense HP4, and CrystaSense HP5, available from Croda Europe Ltd., Yorkshire, England, are satisfactory polyamides for the present invention. These polyamides are believed to be those described in U.S. Pat. No. 8,664,292 to Pavlin et al., the disclosure of which is incorporated herein by reference.

Appropriate carrier solvents include benzyl benzoate, diethyl phthalate, dioctyl adipate, dipropylene glycol, dipropylene glycol methyl ether acetate, mineral oil, isopropyl myri.state [“iPM”], naptha (such as the material sold under the trade name ISOPAR™ G FLUID by ExxonMobil Chemical), low-odor, low-aromatic hydrocarbon solvents (such as the material sold under the trade name ISOPAR™ M FLUID by ExxonMobil Chemical), ethoxylated and propoxylated alcohols (such as the material sold under the trade name SURFONIC® JL 80 X by Huntsman International, The Woodlands, Tex.), jojoba oil, caprylicicapric triglycerides (such as the material sold under the trade name NEOBEE® 1053 by Stepan Company), polyethylene glycol sorbitan monolaurate (polyoxyethylenesorbitan monolaurate), isostearyl isostearate, C12-15 alkyl benzoate, Polyethylene glycol tert-octylphenyl ether (such as the material sold under the trade name Triton™ X-100 by Sigma-Aldrich), triethyl citrate, sorbitan oleate, ethanol, water, propylene glycol, n-hexane, methyl esters, toluene, and polyethylene glycol.

The fragrance can be any appropriate fragrance, preferably selected as a mixture of two or more of the following fragrance families: floral, fruity, citrus, woody, herbaceous, edible, fresh, spice, and edible. Fragrances are complex mixtures of ingredients with widely varying polarities. Fragrances are available from, for example, Belle-Air Fragrances, Inc., Mundelein, Illinois. Exemplary fragrances (with stock numbers from Belle-Fragrances) include:
(1) From the more polar spice fragrance family: Apple Cinnamon (stock number 204377);
(2) From the more non-polar woody fragrance family: Balsam & Cedar (stock number 88793); and
(3) From a more balanced mixed polarity: St. Nick (stock number 202472).

The following examples all produced a transparent melt having a haze value of five percent or less as measured by ASTM D1003-13.

### Example 1

| Ingredient | Weight percent |
|---|---|
| CrystaSense LP1 | 50 |
| IPM | 25 |
| St. Nick | 25 |

CrystaSense LP (100 g) and IPM (50 g) were mixed to 110 C until uniform. The mixture was cooled to 80 C. St. Nick fragrance (50 g) was warmed to 55 C and added to the mixture. The mixture was covered and stirred until uniform. When cooled to about 75 or 80 C, the mixture was poured into molds of about five grams per cavity and cooled to room temperature. This additive has a mixed polarity profile requiring minimal polarity adjustment to meet the transparency and melt index criteria described above.

### Example 2

| Ingredient | Weight percent |
|---|---|
| CrystaSense LP1 | 50 |
| White mineral oil | 25 |
| St. Nick | 25 |

CrystaSense LP (100 g) and white mineral oil (50 g) were mixed to 110 C until uniform. The mixture was cooled to 80 C. St. Nick fragrance (50 a) was warmed to 55 C and added to the mixture, The mixture was covered and stirred until uniform. When cooled to about 75 or 80 C, the mixture was poured into molds of about five grams per cavity and cooled to room temperature. This additive has a mixed polarity profile requiring minimal polarity adjustment to meet the transparency and melt index criteria described above.

### Example 3

| Ingredient | Weight percent |
|---|---|
| CrystaSense LP1 | 50 |
| IPM | 12.5 |
| White mineral oil | 12.5 |
| St. Nick | 25 |

CrystaSense LP (100 g), IPM (25 g), and white mineral oil (25 g) were mixed to 110 C until uniform. The mixture was cooled to 80 C. St. Nick fragrance (50 g) was warmed to 55 C and added to the mixture. The mixture was covered and stirred until uniform. When cooled to about 75 or 80 C, the mixture was poured into molds of about five grams per cavity and cooled to room temperature, This additive has a mixed polarity profile requiring mini polarity adjustment to meet the transparency and melt index criteria described above.

### Example 4

| Ingredient | Weight percent |
|---|---|
| CrystaSense LP1 | 25 |
| Crystasense MP | 25 |
| IPM | 25 |
| St. Nick | 25 |

CrystaSense LP (50 g), Crystasense MP (50 g), and 1PM (50 g) were mixed to 110 C until uniform. The mixture was cooled to 80 C. St. Nick fragrance (50 g) was warmed to 55 C and added to the mixture. The mixture was covered and stirred until uniform. When cooled to about 75 or 80 C. the mixture was poured into molds of about five grams per cavity and cooled to room temperature. This additive has a mixed polarity profile requiring minimal polarity adjustment to meet the transparency and melt index criteria described above.

### Example 5

| Ingredient | Weight percent |
|---|---|
| CrystaSense LP1 | 25 |
| Crystasense MP | 25 |
| White mineral oil | 25 |
| St. Nick | 25 |

CrystaSense LP (50 g), Crystasense MP (50 g), and white mineral oil (50 g) were mixed to 11.0 C until uniform. The mixture was cooled to 50 C. St. Nick fragrance (50 g) was warmed to 55 C and added to the mixture. The mixture was covered and stirred until uniform. When cooled to about 75 or 80 C, the mixture was poured into molds of about five grams per cavity and cooled to room temperature. This additive has a mixed polarity profile requiring minimal polarity adjustment to meet the transparency and melt index criteria described above.

### Example 6

| Ingredient | Weight percent |
|---|---|
| CrystaSense LP1 | 25 |
| Crystasense MP | 25 |
| White mineral oil | 12.5 |
| IPM | 12.5 |
| St. Nick | 25 |

CrystaSense LP (50 g), Crystasense MP (50 g), white mineral oil (25 g) and IPM (25 g) were mixed to 110 C until uniform. The mixture was cooled to 80 C. St. Nick fragrance (50 g) was warmed to 55 C and added to the mixture. The mixture was covered and stirred until uniform, When cooled to about 75 or 80 C, the mixture was poured into molds of about five grams per cavity and cooled to room temperature. This additive has a mixed polarity profile requiring minimal polarity adjustment to meet the transparency and melt index criteria described above.

### Example 7

| Ingredient | Weight percent |
|---|---|
| CrystaSense LP1 | 50 |
| White mineral oil | 25 |
| Balsam & Cedar | 25 |

CrystaSense LP (100 g) and white mineral oil (50 g) were mixed to 110 C until uniform. The mixture was cooled to 80 C. Balsam & Cedar fragrance (50 g) was warmed to 55 C and added to the mixture. The mixture was covered and stirred until uniform. When cooled to about 75 or 80 C, the mixture was poured into molds of about five grams per cavity and cooled to room temperature. This additive has a more non-polar profile, requiring a carrier polarity adjustment to meet the transparency and melt index criteria described above.

### Example 8

| Ingredient | Weight percent |
|---|---|
| CrystaSense LP1 | 50 |
| IPM | 12.5 |
| White mineral oil | 12.5 |
| Apple Cinnamon | 25 |

CrystaSense LP (100 g), IPM (25 g), and white mineral oil (25 g) were mixed to 110 C until uniform. The mixture was cooled to 80 C. Apple Cinnamon fragrance (50 g) was warmed to 55 C and added to the mixture. The mixture was covered and stirred until uniform. When cooled to about 75 or 80 C, the mixture was poured into molds of about five grams per cavity and cooled to room temperature. This additive has a more polar profile requiring a carrier polarity adjustment to meet the transparency and melt index criteria described above.

### Example 9

| Ingredient | Weight percent |
|---|---|
| CrystaSense LP1 | 40 |
| Crystasense MP | 10 |
| IPM | 25 |
| Apple Cinnamon | 25 |

CrystaSense LP (50 g), Crystasense MP (50 g), and IPM (50 g) were mixed to 110 C until uniform. The mixture was cooled to 80 C. Apple Cinnamon fragrance (50 g) was warmed to 55 C and added to the mixture. The mixture was covered and stirred until uniform. When cooled to about 75 or 80 C, the mixture was poured into molds of about five grams per cavity and cooled to room temperature. This additive has a more polar profile requiring a polymer adjustment to meet the transparency and melt index criteria described above.

### Example 10

| Ingredient | Weight percent |
|---|---|
| CrystaSense LP1 | 25 |
| Crystasense MP | 25 |
| White mineral oil | 10 |
| Balsam & Cedar | 40 |

CrystaSense LP (50 g), Crystasense MP (50 g), and white mineral oil (20 g) were mixed to 110 C until uniform. The mixture was cooled to 80 C. Balsam & Cedar fragrance (80 g) was warmed to 55 C and added to the mixture, The mixture was covered and stirred until uniform. When cooled to about 75 or 80 C, the mixture was poured into molds of about five grams per cavity and cooled to room temperature. This additive has a more non-polar profile requiring a polymer adjustment to meet the transparency and melt index criteria described above.

### Example 11

| Ingredient | Weight percent |
|---|---|
| CrystaSense LP1 | 35 |
| Crystasense MP | 15 |
| White mineral oil | 12.5 |
| IPM | 12.5 |
| Balsam & Cedar | 25 |

CrystaSense LP (70 g), Crystasense MP (30 g), white mineral oil (25 g) and 1PM (25 g) were mixed to 110 C until uniform. The mixture was cooled to 80 C. Balsam & Cedar fragrance (50 g) was warmed to 55 C and added to the mixture. The mixture was covered and stirred until uniform. When cooled to about 75 or 80 C, the mixture was poured into molds of about five grams per cavity and cooled to room temperature. This additive has a more non-polar profile requiring polarity balancing of polymer and carrier to meet the transparency and melt index criteria described above.

While preferred embodiments of the present invention are shown and described, it is envisioned that those skilled in the art may devise various modifications of the present invention without departing from the spirit and scope of the appended claims.

We claim:

1. A combination comprising
at least one polyamide polymer;
at least one carrier solvent; and
a polarity-adjusted additive,

2. The combination of claim 1, wherein the additive comprises at least one of a fragrance, a mal-odor counteractant, a therapeutic agent, an insect repellant, a pesticide, and an air freshener.

3. The combination of claim 1, wherein the polyamide polymer comprises at least one of an ester-terminated polyamide, a tertiary-terminated polyamide, a polyalkyleneoxy-terminated polyamide, and a polyether polyamine.

4. The combination of claim 1, wherein the at least one carrier solvent has a solubility parameter in the range between 7.09 and 8.9.

5. The combination of claim 1, wherein the carrier solvent comprises at least one of benzyl benzoate, diethyl phthalate, dioctyl adipate, dipropylene glycol, dipropylene glycol methyl ether acetate, mineral oil, isopropyl myristate, naptha), a low-odor, low-aromatic hydrocarbon solvents, an ethoxylated alcohol, a propoxylated alcohol, jojoba oil, a caprylic triglyceride, a capric triglyceride, polyethylene glycol sorbitan monolaurate, isostearyl isostearate, a C12-15 alkyl benzoate, polyethylene glycol tert-octylphenyl ether, triethyl citrate, sorbitan oleate, ethanol, water, propylene glycol, n-hexane, methyl esters, toluene, and polyethylene glycol.

6. The combination of claim 1, wherein the at least one polyamide polymer comprises 30 to 60 weight percent of the combination.

7. The combination of claim 1, Wherein the at least one carrier solvent comprises 10 to 50 weight percent of the combination.

8. The combination of claim 1, wherein the at least one polarity-adjusted additive comprises 1 to 60 weight percent of the combination.

9. The combination of claim 1, further comprising at least one of a dye and an inert material.
